(19) United States
(12) Patent Application Publication
(10) Pub. No.: US 2026/0216041 A1
(43) Pub. Date: Jul. 30, 2026

(54) IN-SILICO-DESIGNED BOTULINUM TOXIN MIMETIC PEPTIDE FOR INHIBITING RELEASE OF NEUROTRANSMITTERS INCLUDING ACETYLCHOLINE, AND USE THEREOF IN WRINKLE REDUCTION

(71) Applicant: MEDYTOX INC., Cheongju-si, Chungcheongbuk-do (KR)

(72) Inventors: Su Hyeok KIM, Suwon-si, Gyeonggi-do (KR); Hyo Jeong CHOI, Suwon-si, Gyeonggi-do (KR); Min Hee SHIN, Suwon-si, Gyeonggi-do (KR); Dong Kyu LEE, Suwon-si, Gyeonggi-do (KR)

(21) Appl. No.: 19/141,390

(22) PCT Filed: Dec. 21, 2023
(86) PCT No.: PCT/KR2023/021320

§ 371 Date: Jun. 20, 2025

(30) Foreign Application Priority Data
Dec. 21, 2022 (KR) 10-2022-0180689

(51) Int. Cl. A61K 8/66; A61Q 19/02; A61Q 19/08; C12N 9/52

(52) U.S. Cl. CPC A61K 8/66; A61Q 19/02; A61Q 19/08; C12N 9/52; C12Y 304/24069

(57) ABSTRACT

Provided are a peptide that inhibits the secretion of neurotransmitters from cells, and uses thereof.

### TECHNICAL FIELD

The present disclosure relates to a peptide that inhibits the secretion of neurotransmitters from cells, and uses thereof.

### BACKGROUND ART

A SNARE complex is a complex including SNAP-25, syntaxin, and VAMP2 and is involved in neurotransmitter release. The release of neurotransmitters at synapses is associated with the fusion of synaptic vesicles with a neuronal plasma membrane and requires several proteins that act together to form a synaptic fusion complex. These proteins are collectively referred to as SNARE proteins and include SNAP-25, syntaxin, and synaptobrevin. Synaptobrevin is also referred to as VAMP2. While VAMP2 is located on the synaptic vesicle membrane, SNAP-25 and syntaxin are associated with the cell membrane. Calcium release causes the formation of a complex, which bring the synaptic vesicles into proximity with the cell membrane, allowing the cell membranes to fuse. Through the fusion, neurotransmitters included in the vesicles are released into the synapse. The neurotransmitters may include dopamine, acetylcholine, or norepinephrine. Although there is still a need for peptides derived from VAMP2 and capable of efficiently inhibiting the formation of the SNARE complex, these SNARE proteins have the disadvantage of not being able to effectively act on the skin due to the protective function of the skin.

Meanwhile, attempts have been made to deliver drugs into cells by using peptides that act as carriers capable of transporting target proteins into cells to transport drugs or proteins for treatment.

However, there is a need for peptides that have cell permeability even when not fused to these protein transport domains.

### DISCLOSURE OF INVENTION

### Technical Problem

One aspect provides a peptide or a pharmaceutically acceptable salt thereof that inhibits the secretion of neurotransmitters from neurons.

Another aspect provides a polynucleotide encoding the peptide, and a vector and a host cell including the same.

Another aspect provides a composition or kit including the peptide for use in skin whitening or wrinkle improvement.

Another aspect provides a method of skin whitening or wrinkle improvement in a subject, by administering the peptide to the subject.

### Solution to Problem

One aspect provides a peptide that inhibits the secretion of a neurotransmitter in a neuron, or a pharmaceutically acceptable salt thereof, wherein the peptide has the amino acid sequence of SEQ ID NO: 1.

The amino acids of the above peptide may be D- or L-amino acids. An N-terminus or C-terminus of the above peptide may be modified. The above modification may be a chemical modification. The above modifications may aid in the stability, safety or delivery of the peptide. The above modification may not substantially reduce the physiological activity of the peptide. For example, the N-terminus of the peptide may be bound to any one protecting group selected from the group consisting of an acetyl group, a fluorenylmethoxycarbonyl group, a formyl group, a palmitoyl group, a myristyl group, a stearyl group, a butoxycarbonyl group, an allyloxycarbonyl group, and polyethylene glycol (PEG). For example, the C-terminus of the peptide may be bound to any one protecting group selected from the group consisting of an amino group (—NH₂), a tertiary alkyl group, and an azide (—NHNH₂). The above modification may include esterification of the carboxyl group of glutamic acid and aspartic acid. By the above modification, a negative charge of the amino acid may be removed and the hydrophobicity may be increased. Accordingly, modified peptides may have increased bioavailability, including stability and fat solubility, and may be able to readily pass through the blood-brain barrier and/or epithelial tissue. The N-terminus of the above peptide may be acetylated. The C-terminus of the above peptide may be aminated. The above modification may be hydrolyzed by intracellular esterases, etc. in the body.

The above peptide may inhibit neurotransmitter release by inhibiting the formation of a SNARE complex including SNAP-25, syntaxin, and VAMP2. The above SNARE complex may be, for example, a complex including SNAP-25, syntaxin 1a, and VAMP2. Release of neurotransmitters at synapses is associated with the fusion of synaptic vesicles with a neuronal plasma membrane and requires several proteins that act together to form a synaptic fusion complex. These proteins are collectively referred to as SNARE proteins and include SNAP-25, syntaxin, and synaptobrevin. Synaptobrevin is also referred to as VAMP2. While VAMP2 is located on the synaptic vesicle membrane, SNAP-25 and syntaxin are associated with the cell membrane. Calcium release causes the formation of a complex, which bring the synaptic vesicles into proximity with the cell membrane, allowing the cell membranes to fuse. Through the above fusion, neurotransmitters included in the vesicles are released into the synapse. The above neurotransmitters include any that are released through synapses. The neurotransmitter may be dopamine, acetylcholine, epinephrine, norepinephrine, serotonin, histamine, glutamate, glycine, gamma-aminobutyric acid or a combination thereof. The neurotransmitter may be, for example, dopamine, acetylcholine, or a combination thereof. The above peptide mimics VAMP2 and inhibits the formation of SNARE complexes, thereby inhibiting neurotransmitter release into synapses. Accordingly, the peptide may alleviate or improve various symptoms caused by the release of the neurotransmitter into the synapse. The above peptide may prevent or treat various symptoms caused by the release of the neurotransmitter into the synapse. The above symptoms may include blepharospasm, facial spasm, hemifacial spasm, strabismus, urinary incontinence, polyuria, equinus deformity caused by pediatric cerebral palsy, hyperhidrosis, headache, migraine, upper extremity muscle stiffness, myoclonus, spastic torticollis, febrile seizures, cerebral palsy, temporomandibular disorder, neuralgia, myalgia, or dystonia. The above peptides may whiten skin or improve wrinkles. Additionally, the peptides may be used to improve or treat neuron-exocytosis mediated symptoms. The above symptoms may be spastic ailments. The above spastic ailments may be dystonias, strabismus, tics, blepharospasm, or facial scoliosis.

The above peptide may be obtained through classical solid-phase chemical peptide synthesis methods. Additionally, the peptide may be obtained by a method based on recombinant DNA technology. For example, the method may include introducing a polynucleotide encoding the peptide into an appropriate plasmid or vector, introducing the same into a host cell, culturing the resulting cell such that the peptide is formed in the culture, and optionally purifying the peptide.

The above peptide may or may not be fused to a membrane permeable peptide. The above cell membrane permeable peptide may be one that increases passage of the fused peptide through the cell membrane. The above cell membrane permeable peptide may be selected from the group consisting of TD1, IMT-P8, Transkin, VP2-2, RBD-1, SN25-2, STX-1, and TDb-1.

In the present application, the peptide is understood to include not only the peptide but also a salt thereof. The above salt may be a pharmaceutically or cosmetically acceptable salt of the above peptide.

Another aspect provides a composition for use in skin whitening or wrinkle improvement including the above peptide as an active ingredient.

The composition may further include a pharmaceutically or cosmetically acceptable carrier. The above carrier may be a diluent or an excipient. The above carrier may be an antioxidant, stabilizer, solubilizer, vitamin, pigment, fragrance, etc. The diluent may be water, a buffer, or saline.

Additionally, the composition may further include a pharmaceutically or cosmetically acceptable adjuvant.

The above composition may be a cosmetic or pharmaceutical or food composition. The composition may be in any one formulation, for example, a solution, a suspension, an emulsion, a paste, a gel, a cream, a lotion, a powder, a soap, a surfactant-containing cleanser, an oil, a powder foundation, an emulsion foundation, a wax foundation, and a spray.

When the above formulation is a paste, cream, or gel, it may include one or more of animal oil, vegetable oil, wax, paraffin, starch, tragacanth, cellulose derivatives, polyethylene glycol, silicone, bentonite, silica, talc, and zinc oxide as a carrier component.

When the above formulation is a powder or spray, the carrier may be one or more of lactose, talc, silica, aluminum hydroxide, calcium silicate, and polyamide powder. Especially when it is a spray, the composition may further include a propellant such as chlorofluorohydrocarbons, propane/butane and dimethyl ether.

When the above formulation is a solution or emulsion, the carrier may include one or more of a solvent, a solubilizer and an emulsifier. The carrier may be, for example, one or more of ethanol, isopropanol, ethyl carbonate, ethyl acetate, benzyl alcohol, benzyl benzoate, propylene glycol, 1,3-butylglycol oil, glycerol aliphatic ester, polyethylene glycol and fatty acid esters of sorbitan.

When the above formulation is a suspension, the carrier may be one or more of a liquid diluent such as water, ethanol and propylene glycol, a suspending agent such as ethoxylated isostearyl alcohol, polyoxyethylene sorbitol ester and polyoxyethylene sorbitan ester, microcrystalline cellulose, aluminum methahydroxide, bentonite, agar and tragacanth.

The content of the above peptide may be an effective amount for skin whitening or improving wrinkles. The content may be, for example, 0.001 to 95.0%, 0.001 to 70.0%, 0.001 to 50.0%, or 0.01 to 50.0% based on the weight of the composition.

Another aspect provides a kit for use in skin whitening or wrinkle improvement including the above peptide.

The kit may further include one or more selected from the group consisting of a carrier and an adjuvant. Additionally, the kit may further include instructions describing a process for using the peptide for skin whitening or improving wrinkles.

Another aspect provides a method of skin whitening or improving wrinkles in a subject including administering to the subject an amount of the peptide described above effective to skin whitening or improving wrinkles.

The above peptide may be in the form of itself or of the above composition. The above administration may be administered by any route. The above administration may be oral or parenteral. The above administration may be by application to the nasal cavity, mucous membranes or skin. The above subject may be a human or a non-human animal, for example a mammal. The above method may be a method of makeup application.

Another aspect provides a polynucleotide encoding the above-mentioned peptide.

Another aspect provides a vector including the polynucleotide. The above vector may include any one that is used to deliver the above polynucleotide. The vector may include a nucleic acid construct, a plasmid, or a virus-derived vector. The above vector may be an expression vector, for example, an expression vector capable of being expressed in a mammal.

Another aspect provides a recombinant host cell including the polynucleotide. The host cell may be a bacterial or animal cell. The above animal cell may be a known animal cell such as a Chinese hamster ovary cell (CHO).

Another aspect provides a composition for use in inhibiting the formation of a SNARE complex including SNAP-25, syntaxin and VAMP2, including the above peptide as an active ingredient. The composition may further include a carrier or adjuvant. The composition may be a cosmetic, pharmaceutical, or food composition.

The composition may be intended to improve or treat neuron-exocytosis mediated symptoms. The above symptoms may include blepharospasm, facial spasm, hemifacial spasm, strabismus, urinary incontinence, polyuria, equinus deformity caused by pediatric cerebral palsy, hyperhidrosis, headache, migraine, upper extremity muscle stiffness, myoclonus, spastic torticollis, febrile seizures, cerebral palsy, temporomandibular disorder, neuralgia, myalgia, or dystonia. The above symptoms may be spastic ailments. The above spastic ailments may be dystonias, strabismus, tics, blepharospasm, or facial scoliosis.

The content of the above peptide and the carrier or adjuvant are as described above.

Another aspect provides a kit for use in inhibiting the formation of a SNARE complex including SNAP-25, syntaxin and VAMP2 including the above peptides. The above kit may further include one or more of a carrier and an adjuvant.

Another aspect provides a method of inhibiting the formation of a SNARE complex in a subject including administering to the subject a peptide as described above.

The above method may be for improving or treating neuron-exocytosis mediated symptoms. The above symptoms may include blepharospasm, facial spasm, hemifacial spasm, strabismus, urinary incontinence, polyuria, equinus deformity caused by pediatric cerebral palsy, hyperhidrosis, headache, migraine, upper extremity muscle stiffness, myoclonus, spastic torticollis, febrile seizures, cerebral palsy, temporomandibular disorder, neuralgia, myalgia, or dystonia. The above symptoms may be spastic ailments. The above spastic ailments may be dystonias, strabismus, tics, blepharospasm, or facial scoliosis.

The above peptide may be in the form of a peptide or a composition including the peptide. The amount of the peptide administered may be an amount effective to inhibit the formation of a SNARE complex in the subject.

### Advantageous Effects of Invention

According to an aspect, a peptide may be used for skin whitening or wrinkle improvement.

According to an aspect, a polynucleotide encoding the peptide, a vector including the same, and a host cell may be used to produce the peptide.

According to an aspect, a composition or kit including the peptide may be used for skin whitening or wrinkle improvement, or improving or treating neuron-exocytosis mediated symptoms.

According to an aspect, a method may allow to effectively improve or treat skin whitening or wrinkle improvement of an individual, or neuron-exocytosis mediated symptoms.

### BRIEF DESCRIPTION OF DRAWINGS

FIG. 1 is a diagram illustrating an effect of P631 peptide on dopamine secretion in PC12 cells.

FIG. 2 is a diagram illustrating an effect of P631 peptide on acetylcholine secretion in PC12 cells.

FIG. 3 is a diagram illustrating the results of a cytotoxicity test of the P631 peptide.

FIG. 4 is a diagram illustrating the results of measuring the effect of P631 peptide on skin irritation.

FIG. 5 is a diagram illustrating the results of culturing cells in the presence of FITC-P631 (red) or FITC-control group peptide (black) and measuring absorbance at 470 nm corresponding to FITC emission.

### BEST MODE FOR CARRYING OUT THE INVENTION

The present disclosure will be described in more detail below with reference to embodiments. However, these embodiments are intended to illustrate the present disclosure by way of example and the scope of the invention is not limited to these embodiments.

### Example 1: Screening of Peptides with Neurotransmitter Secretion Inhibition Ability and Confirmation of their Efficacy

In neurons, a SNARE complex is formed, and neurotransmitters such as acetylcholine are released extracellularly and bind to receptors on muscle cells, thereby transmitting a stimulus. This stimulation induces contraction of muscle cells, which results in the formation of facial wrinkles. Here, blocking the formation of the SNARE Complex may prevent muscle contraction and eliminate wrinkles. For example, botulinum toxin is known to eliminate wrinkles by blocking the formation of SNARE complexes and thereby preventing muscle contraction. Botulinum toxin cleaves specific regions of proteins constituting the SNARE complex, thereby inhibiting normal complex formation. Accordingly, if the formation of the SNARE complex is inhibited using a peptide, a wrinkle improvement effect may be exhibited.

In the present example, a peptide that inhibits the formation of the SNARE complex was selected, and its efficacy in inhibiting neurotransmitter secretion, wrinkle improvement, and skin whitening was confirmed.

### 1. Synthesis of Candidate Peptides

In the present example, a polypeptide was synthesized in which the N-terminus of the amino acid sequence of SEQ ID NO: 1 was protected with an acetyl group and the C-terminus was protected with an amino group. The amino acid sequence of SEQ ID NO: 1 was designed according to an in silico-based model. The above polypeptide is also referred to as ‘P631’ below. The above polypeptide was created by modifying a portion of the v-snare coiled-coil domain of VAMP2, which forms the SNARE complex, as a template. The above polypeptide may permeate neurons and control neurotransmitter release. The above polypeptide was prepared through chemical synthesis and used for experiments. The above P631 is not linked to a separate cell permeable peptide.

### 2. Measurement of Neurotransmitter Secretion Inhibition Capacity

It was confirmed whether the P631 peptide inhibits the formation of the SNARE complex and thereby inhibits the secretion of neurotransmitters. The neurotransmitters selected above were dopamine and acetylcholine.

### (1) Effect of P631 Peptide on Dopamine Secretion in Neurons

PC12 cells (Korean Cell Line Bank) were cultured in a 48-well plate coated with Matrigel for 7 days at 37° C. in RPMI1640 medium including 10% FBS and NGF (2.5S; Alomone Labs, N-100) at 50 mg/mL. Afterwards, they were cultured at 37° C. for an additional 3 days in RPMI1640 medium containing 5% FBS. PC12 cells are a cell line derived from a pheochromocytoma that develops in the adrenal medulla of a rat. Because chromaffin cells, which secrete catecholamines in the adrenal medulla, are already differentiated and do not divide, cultured PC12 cells are commonly used to study chromaffin cells.

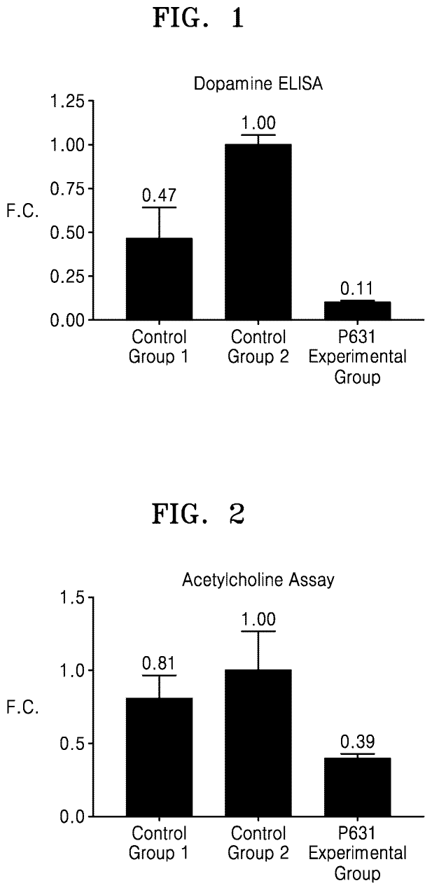

After the culture was completed, RPMI1640 medium alone (Control group 1 and Control group 2 in FIG. 1) or RPMI1640 containing 20 μM P631 peptide was added to PC-12 cells and cultured at 37° C. for 2 hours. Thereafter, the medium was removed, and all wells were washed three times with Krebs buffer solution containing low concentration potassium ions (118 mM NaCl, 1.2 mM MgSO₄, 2.5 mM CaCl₂), 5 mM KCl, 24 mM NaHCO₂, 2 mM KH₂PO₄, and 11 mM dextrose, pH 7.4). Afterwards, 0.1 mL/well of low-potassium ion-containing Krebs buffer solution was added to the peptide-free non-depolarized group (Control group 1 in FIG. 1), and 0.1 mL/well of high-potassium ion-containing Krebs buffer solution (56 mM NaCl, 1.2 mM MgSO₄, 2.5 mM CaCl₂), 68 mM KCl, 24 mM NaHCO₃, 2 mM KH₂PO₄, and 11 mM dextrose, pH 7.4) was added to the peptide-free depolarized group (Control group 2 in FIG. 1) and the P631 peptide-treated depolarized group (Experimental group in FIG. 1), and depolarization was induced by leaving it at 37° C. for 8 minutes. The supernatant of cells that had completed depolarization was collected at 0.315 mL/well and transferred to a new 48-well, and the amount of dopamine secreted into the medium was measured by ELISA according to the manual of the Dopamine ELISA kit (Abnova, KA1887).

The results are shown in FIG. 1. FIG. 1 is a diagram illustrating an effect of P631 peptide on dopamine secretion in PC12 cells. As shown in FIG. 1, the dopamine secretion rate of the peptide-untreated depolarized group, in other words, Control group 2, and the P631 peptide-treated depolarized group, in other words, experimental group, was 100% and 11%, respectively. This indicates that the P631 peptide inhibits dopamine secretion in depolarized neurons.

### (2) Effect of P631 Peptide on Acetylcholine Secretion in Neurons

PC12 cells (Korean Cell Line Bank) were cultured in a 48-well plate coated with Matrigel for 7 days at 37° C. in RPMI1640 medium including 10% FBS and NGF (2.5S; Alomone Labs, N-100) at 50 mg/mL.

After the culture was completed, RPMI1640 medium alone (Control group 1 and Control group 2 in FIG. 2) or RPMI1640 containing 20 μM P631 peptide was added to PC-12 cells and cultured at 37° C. for 2 hours. Thereafter, the medium was removed, and all wells were washed three times with Krebs buffer solution containing low concentration potassium ions (118 mM NaCl, 1.2 mM MgSO₄, 2.5 mM CaCl₂), 5 mM KCl, 24 mM NaHCO₂, 2 mM KH₂PO₄, and 11 mM dextrose, pH 7.4). Afterwards, 0.1 mL/well of low-potassium ion-containing Krebs buffer solution was added to the peptide-free non-depolarized group (Control group 1 in FIG. 2), and 0.1 mL/well of high-potassium ion-containing Krebs buffer solution (56 mM NaCl, 1.2 mM MgSO₄, 2.5 mM CaCl₂), 68 mM KCl, 24 mM NaHCO₃, 2 mM KH₂PO₄, and 11 mM dextrose, pH 7.4) was added to the peptide-free depolarized group (Control group 2 in FIG. 2) and the P631 peptide-treated depolarized group (Experimental group in FIG. 2), and depolarization was induced by leaving it at 37° C. for 8 minutes. The supernatant of cells that had completed depolarization was collected at 0.07 mL/well and transferred to a new 48-well, and the amount of acetylcholine secreted into the medium was measured by ELISA according to the manual of the Acetylcholine ELISA kit (AAT bioquest, 111403).

The results are shown in FIG. 2. FIG. 2 is a diagram illustrating an effect of P631 peptide on acetylcholine secretion in PC12 cells. As shown in FIG. 2, the acetylcholine secretion rate of the peptide-untreated depolarized group, in other words, Control group 2, and the P631 peptide-treated depolarized group, in other words, experimental group, was 100% and 39%, respectively. This indicates that the P631 peptide inhibits acetylcholine secretion in depolarized neurons.

### 2. Confirmation of Toxicity of P631 Peptide

### (1) Raw Material Toxicity Test: MTT Analysis

HDF (Human Dermal Fibroblasts) cells purchased from the Korean Cell Line Bank were seeded at a density of 8×10³ cells/well in 96-well plates in 100 μL of DMEM medium containing 10% FBS and cultured for 24 hours. HDF cells are human-derived fibroblasts. Afterwards, the P631 peptide was diluted in DMEM culture medium at concentrations of 0, 1, 5, 10, 15, 20, 25, 30, 40, and 50 μM, replaced, and cultured for 24 hours.

After 24 hours, 20 μL of MTT solution was added to the DMEM medium to obtain a final MTT concentration of 0.5 mg/mL. After addition of MTT, cells were cultured in a 5% CO₂ incubator at 37° C. for 4 hours. The plate was centrifuged at 1,000 rpm for 10 minutes, and the MTT solution was removed. Each well was washed twice with PBS, and 200 μl of DMSO was added to dissolve the produced formazan. The above plate was placed into a Spectra MAX i3 instrument, and the absorbance at 590 nm was measured for the sample in each well.

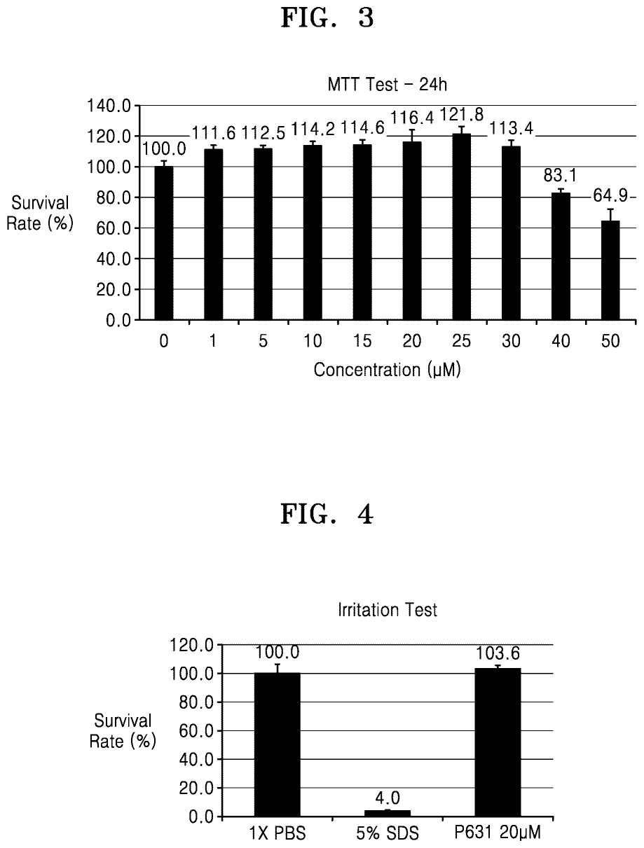

The results are shown in FIG. 3. FIG. 3 is a diagram illustrating the results of a cytotoxicity test of the P631 peptide. As shown in FIG. 3, the P631 peptide showed no cytotoxicity at the experimental concentration of 20 μM, with a cell viability rate of about 80% or more up to 40 μM compared to the untreated group (0 μM) in the MTT assay.

### (2) Skin Irritation Test

A negative control group and a positive control group were prepared by evenly applying 1×PBS and 5% SDS, respectively, to artificial skin (Neoderm®-ED) purchased from Tego Science. The experimental group was evenly applied with 20 μl of 20 μM P631 peptide. After reacting at room temperature for 15 minutes, the mixture was transferred to a well of a 12-well culture plate and incubated for 15 minutes in a 37° C., 5% CO₂ incubator. The artificial skin after the reaction was washed twice with PBS and transferred to a well of a new 12-well culture plate and cultured for 42 hours. After the culture was completed, 2 ml of 0.3 mg/ml MTT solution was added and the cells were cultured for 3 hours. The sample in each well of the above plate was decolorized by adding 0.04 N HCl-isopropanol, placed in Spectra MAX i3, and the absorbance of the sample in each well was measured at 580 nm.

The results are shown in FIG. 4. FIG. 4 is a diagram illustrating the results of measuring the effect of P631 peptide on skin irritation. As shown in FIG. 4, the P631 peptide was non-irritating with a cell viability of about 103.6%. Here, if the cell viability was 50% or more compared to the negative control group, it was determined as non-irritant.

### 3. Cell Permeability Test

NTERA-2 cell line purchased from the Korean Cell Line Bank was differentiated into neuron-like cells. NTERA-2 cell line is a pluripotent human embryonal carcinoma cell line. Differentiated cells were seeded in 6-well plates at a concentration of 1.5×10⁶ cells/well in 2 mL of DMEM/F12 medium containing FBS and B-27 and cultured for 24 hours. FITC was fused to the N-terminus of the P631 peptide and the wild-type peptide of VAMP2. The above peptides are referred to as ‘FITC-P631’ and ‘FITC-control group peptide’, respectively. The existing medium in the 6-well plate was removed, and the two peptides were prepared at 20 UM each in DMEM/F12 medium, 2 mL per well, and cultured for 24 hours. After culture, the existing medium was removed, and the cells were washed using DPBS. Afterwards, the cells were then detached using 0.25% trypsin-EDTA and DMEM/F12 medium. Afterwards, the cells were dissolved in DPBS (FACS buffer) containing 2% FBS and FACS analysis was performed.

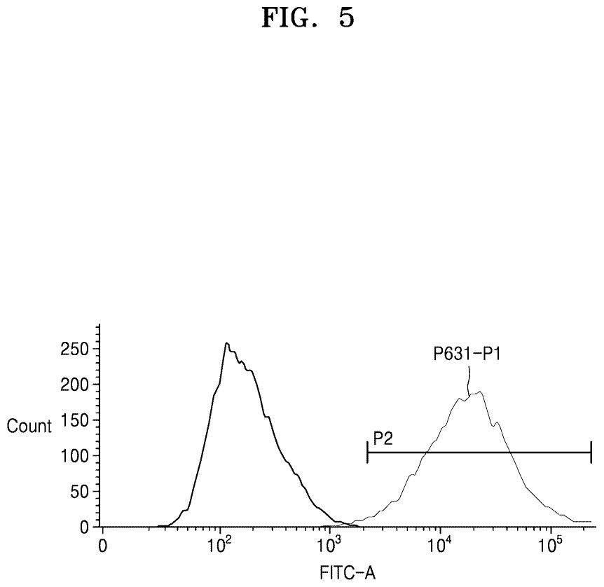

FIG. 5 is a diagram illustrating the results of culturing cells in the presence of FITC-P631 (red) or FITC-control group peptide (black) and measuring the absorbance at 470 nm corresponding to FITC emission. As shown in FIG. 5, the polypeptide-FITC had a significantly higher fluorescence intensity than the scrambled peptide-FITC. The part expressed as ‘P2’ here was determined to be the cell in which FITC was discovered. This indicates that, the P631 peptide may permeate cells better than the control group peptide. In FIG. 5, the vertical axis represents the number of cells, and the horizontal axis represents the intensity of FITC. P631-P1 represents P631.

Sequence Information

SEQ ID NO: 1 (P631):
NRRLRLTAAKLLDLLAIFR
SEQ ID NO: 2-Control group peptide (VAMP2 wild-type peptide):
NRRLQQTQAQVDEVVDIMR

1. A peptide that inhibits secretion of neurotransmitters from neurons, wherein the peptide has the amino acid sequence of SEQ ID NO: 1.

2. The peptide of claim 1, not linked to a cell permeable peptide.

3. The peptide of claim 1, wherein an N-terminus of the peptide is bonded to any one protecting group selected from the group consisting of an acetyl group, a fluorenylmethoxycarbonyl group, a formyl group, a palmitoyl group, a myristyl group, a stearyl group, a butoxycarbonyl group, an allyloxycarbonyl group, and polyethylene glycol (PEG).

4. The peptide of claim 1, wherein a C-terminus of the peptide is bonded to any one protecting group selected from the group consisting of an amino group (—NH2), a tertiary alkyl group, and a hydrozino group (—NHNH2).

5. The peptide of claim 1, wherein the neurotransmitters are dopamine, acetylcholine, epinephrine, norepinephrine, serotonin, histamine, glutamate, glycine, gamma-aminobutyric acid or a combination thereof.

6. A composition, comprising the peptide according to claim 1 as an active ingredient.

7. (canceled)

8. The composition of claim 6, further comprising one or more selected from the group consisting of a diluent, an excipient, a carrier, and an adjuvant.

9. A method of skin whitening or wrinkle improvement, comprising administering to a subject the peptide according to claim 1 in an amount effective for skin whitening or wrinkle improvement.

10. The method of claim 9, wherein the method is for makeup application.


SEQUENCE LISTING (2 sequences)

SEQ ID NO: 1 - protein, 19 aa, synthetic construct
  source 1..19: note = Control group peptide (VAMP2 wild-type peptide)
NRRLRLTAAKLLDLLAIFR

SEQ ID NO: 2 - protein, 19 aa, Homo sapiens
NRRLQQTQAQVDEVVDIMR